(19) United States
(12) Patent Application Publication
(10) Pub. No.: US 2005/0222114 A1
(43) Pub. Date: Oct. 6, 2005

(54) Use of new etonogestrel esters

(76) Inventors: Henrik H. De Nijs, Oss (NL); H. A. A. Van Der Voort, Oss (NL); Dirk D. Leysen, Oss (NL); A. J. Grootenhuis, Oss (NL); J. Van Der louw, Oss (NL)

Correspondence Address: AKZO NOBEL INC.;INTELLECTUAL PROPERTY DEPARTMENT, 7 LIVINGSTONE AVENUE, DOBBS FERRY, NY, 10522-3408, US

(21) Appl. No.: 10/517,362

(22) PCT Filed: May 22, 2003
(86) PCT No.: PCT/EP03/50188

§ 371 Date: Nov. 30, 2004

(30) Foreign Application Priority Data
May 30, 2002 (EP) 02077118.4

(51) Int. Cl. A61K 31/56

(52) U.S. Cl. 514/182

(57) ABSTRACT

The subject invention provides a contraceptive and/or HRT kit comprising a contraceptively and/or therapeutically effective amount of a long-acting etonogestrel ester with a fatty-chain length of C7-C15.

### FIELD OF THE INVENTION

The subject invention concerns the field of (male and female) contraception, (male and female) hormone replacement therapy (HRT) and treatment/prevention of gynaecological disorders.

### BACKGROUND

Contraceptive methods for men and women are important for worldwide reproductive health.

However, no effective and efficient methods of male contraception are as of yet available.

Male contraception seeks to suppress spermatogenesis through the suppression of the gonadotropins luteinizing hormone (LH) and follicle-stimulating hormone (FSH). This results in a depletion of intratesticular testosterone and cessation of spermatogenesis.

Administration of progestagen results in a dose dependent suppression of pituitary gonadotrophins and consequently, a decrease in testosterone levels and a reversible inhibition of spermatogenesis. An exogenous androgen is required to compensate for the reduced testosterone levels. In the same way, male HRT can be accomplished, resulting in replacement of testosterone by an exogenous androgen which is safer on the prostate than endogenous testosterone.

The use of progestogens together with androgens for use as male contraceptives and HRT is known (Guerin and Rollet (1988), International Journal of Andrology 11, 187-199).

However, the use of specific esters of etonogestrel for male contraception and HRT has not been suggested.

In addition, the use of progestogens together with estrogens for use in female contraception and HRT is known (M. Tausk, J. H. H. Thijssen, Tj. B. van Wimersma Greidanus, “Pharmakologie der Hormone”, Georg Thieme Verlag, Stuttgart, 1986).

Progestagens are widely used for female contraception and in female HRT. In contraception, the combination progestagen-estrogen oral contraceptives are the most widely used. Administration of such a combination results in a number of effects: it blocks ovulation, it interferes with phasic development of the endometrium which decreases the chance for successful implantation, and it causes the cervical mucus to become so viscous that it hinders sperm penetration. Most progestagen-only-pills (POP's) aim at the last mentioned effect only.

Female HRT is aimed at suppletion of endogenous estrogen for the treatment of peri- and postmenopausal complaints (hot flushes, vaginal dryness), and for prevention of symptoms of long-term estrogen deficiency. The latter include osteoporosis, coronary artery disease, urogenital incontinence, and possibly also Alzheimer's disease and colorectal cancer. A drawback of long-term unopposed estrogen administration is the associated increase in endometrium proliferation, which in turn may increase the risk of endometrial cancer. For that reason, progestagens are co-administered in long-term regimes, because of their ability to reduce the proliferative activity of endometrial epithelium and to induce secretory conversion.

However, the use of specific esters of etonogestrel for female contraception, female HRT and treatment/prevention of gynaecological disorders has not been suggested.

The subject invention describes esters of etonogestrel with a fatty-chain length of C7-C15, preferably C10-C12, which have a good pharmacokinetic profile and enable a single-dose administration of a progestogen with a long duration of action.

### SUMMARY OF THE INVENTION

The subject invention provides a male and female contraceptive and/or HRT kit comprising a contraceptively and/or therapeutically effective amount of a long-acting etonogestrel ester with a fatty-chain length of C7-C15, preferably C10-C12.

In addition, the use of these esters for treatment and prevention of female gynaecological disorders such as endometriosis, menorrhagia, meno-metrorrhagia, pre-menstrual syndrome and dysmenorrhoea are also contemplated.

### FIGURES

FIG. 1 Chemical structures of etonogestrel heptanoate (etonogestrel enanthate), etonogestrel nonanoate, etonogestrel decanoate, etonogestrel undecanoate, etonogestrel dodecanoate, etonogestrel tridecanoate, and etonogestrel pentadecanoate.

FIG. 2a Effect of one intramuscular (IM) injection of etonogestrel, etonogestrel heptanoate (etonogestrel enanthate), etonogestrel nonanoate and etonogestrel undecanoate on plasma levels of etonogestrel in male intact rabbits. Means and SEM of N=3.

FIG. 2b Effect of one intramuscular (IM) injection of etonogestrel heptanoate (etonogestrel enanthate), etonogestrel nonanoate, etonogestrel decanoate, etonogestrel undecanoate, etonogestrel dodecanoate, etonogestrel tridecanoate on plasma levels of etonogestrel in male intact rabbits. Means and SEM of N=3.

FIG. 3 Chemical structure of MENT-undecanoate, MENT-buciclate, testosterone heptanoate (testosterone enanthate) and testosterone undecanoate.

FIG. 4 Time dependent effects of one s.c injection of 20 mg/kg of MENT-undecanoate (MENT-U), MENT-buciclate (MENT-B), testosterone-heptanoate (testosterone-enanthate, TE) and testosterone-undecanoate (TU) in castrated male rabbits on serum MENT or testosterone (T). Results are means of N=3.

FIG. 5 Pharmacokinetics of testosterone enanthate, testosterone undecanoate and testosterone buciclate after one IM injected in male hypogonadal men with indicated doses on the plasma levels of serum testosterone. Normal range of serum testosterone is indicated with a dashed line. Derived from E. Nieschlag and H. M. Behre. Testosterone Therapy. In: Andrology, Male reproductive health and dysfunction., edited by E. Nieschlag and H. M. Behre, Berlin, Heidelberg and New York:Springer-Verlag, 1997, p. 297-309.

### DETAILED DESCRIPTION OF THE INVENTION

The subject invention provides a contraceptive and/or HRT kit comprising a contraceptively and/or therapeutically effective amount of a long-acting etonogestrel ester with a fatty-chain length of C7-C15, preferably C10-C12.

In one embodiment, the kit further comprises a contraceptively and/or therapeutically effective amount of an androgen ester, preferably MENT undecanoate.

In another embodiment, the kit further comprises a contraceptively and/or therapeutically effective amount of an estrogen, such as mestranol, ethynylestradiol, ethynylestradiol sulfonate estradiol, estradiol valerate, estriol, estriol succinate, quinestrol, estropipate, sodium estrone sulfate or sodium equilin sulfate.

The subject invention further contemplates a use of a contraceptively and/or therapeutically effective amount of a long-acting etonogestrel ester with a fatty-chain length of C7-C15, preferably C10-C12, for the preparation of a medicament for contraception and/or HRT.

In one embodiment, the etonogestrel ester is used in conjunction with a contraceptively and/or therapeutically effective amount of a long-acting androgen ester for the preparation of a medicament for male contraception and/or male HRT. The long-acting androgen ester is preferably MENT undecanoate.

In another embodiment, the etonogestrel ester is used in conjunction with a contraceptively and/or therapeutically effective amount of an estrogen for the preparation of a medicament for female contraception and/or female HRT and/or for the treatment and/or prevention of a female gynaecological disorder such as endometriosis, menorrhagia, meno-metrorrhagia, pre-menstrual syndrome and dysmenorrhoea.

The subject invention also encompasses a method of contraception and/or HRT comprising administering to a subject a contraceptively and/or therapeutically effective amount of a long-acting etonogestrel ester with a fatty-chain length of C7-C15, preferably C10-C12.

In one embodiment, a contraceptively and/or therapeutically effective amount of a long-acting androgen ester, preferably MENT undecanoate, is administered in conjunction with the etonogestrel ester.

In another embodiment, a contraceptively and/or therapeutically effective amount of an estrogen is administered in conjunction with the etonogestrel ester.

The subject invention further provides a method of treating and/or preventing a female gynaecological disorder comprising administering to a female subject a therapeutically effective amount of a long-acting etonogestrel ester with a fatty-chain length of C7-C15, preferably C10-C12, effective to treat and/or prevent the disorder.

In the above aspects of the subject invention, the contraceptively and/or therapeutically effective amount of MENT undecanoate is 50-400 mg and the contraceptively and/or therapeutically effective amount of etonogestrel ester is 25-200 mg.

In a preferred embodiment, the contraceptively and/or therapeutically effective amount of MENT undecanoate is 50-200 mg and the contraceptively and/or therapeutically effective amount of etonogestrel ester is 50-100 mg.

In a specifically preferred embodiment, the contraceptively and/or therapeutically effective amount of MENT undecanoate is 100 mg and the contraceptively and/or therapeutically effective amount of etonogestrel ester is 50 mg.

The progestogen and testosterone esters can be prepared by dissolving it in a suitable amount of an oily medium, such as arachis oil, oleic acid, castor oil, ethyl undecanoate, almond oil, sesame oil, coconut oil, olive oil, soyabean oil, (purified) triglycerised, propylene glycol esters, ethyl oleate and the like, including mixtures of oils. The amount of esters that can be dissolved differs per chosen medium, but will generally be within the range of from 100-400 mg. The preferred oil is arachis oil or ethyl undecanoate.

Additives common to injection fluids can be added to the solution if desired. Suitable additives are known to the person skilled in the art. Possible additives include liquids that serve to lower the viscosity of the formulation, e.g. benzyl alcohol, benzyl benzoate, benzyl propionate, ethyl oleate or ethyl undecanoate.

The compounds of the subject invention can principally be administered via any suitable route available to the skilled person.

In the case of oral administration, a solid dosage unit such as a tablet or a capsule is contemplated. The compounds of the invention can be formulated with a pharmaceutically acceptable carrier, such as described in the standard reference, Gennaro et al, Remmington: The Science and Practice of Pharmacy, (20th ed., Lippincott Williams & Wilkins, 2000, see especially Part 5: Pharmaceutical Manufacturing). The compounds of the invention and the pharmaceutically acceptable carrier may be compressed into solid dosage units, such as pills, tablets, or be processed into capsules or suppositories. By means of pharmaceutically suitable liquids the compounds can also be applied as an injection preparation in the form of a solution, suspension, emulsion, or as a spray, e.g. nasal spray. For making dosage units, e.g. tablets, the use of conventional additives such as fillers, colorants, polymeric binders, lubricants, flow enhancers, glidants and the like is contemplated. In general any pharmaceutically acceptable additive which does not interfere with the function of the active compounds can be used. The compounds of the invention may also be included in an implant, a vaginal ring, a patch, a gel, and the like.

Suitable carriers with which the compositions can be administered include lactose, starch, cellulose derivatives and the like, or mixtures thereof used in suitable amounts.

The dose of and regimen of administration of the compounds of the invention, or a pharmaceutical composition thereof, to be administered will depend on the therapeutic effect to be achieved and will vary with the route of administration, and the age and condition of the individual subject to whom the medicament is to be administered, and/or the particular contraceptive or HRT regimen in which it is used. Typical dosage amounts are 0.001-5 mg per kg body weight.

The present invention is further described in the following examples which are not in any way intended to limit the scope of the invention as claimed.

### EXAMPLES

### Example 1

### Kinetics of Etonogestrel C7, C9, C10, C11, C12 and C13 Esters in Rabbits

The following etonogestrel esters were prepared and tested in rabbits:
Etonogestrel heptanoate
Etonogestrel nonanoate
Etonogestrel decanoate
Etonogestrel undecanoate
Etonogestrel dodecanoate
Etonogestrel tridecanoate

Etonogestrel pentadecanoate was also prepared.

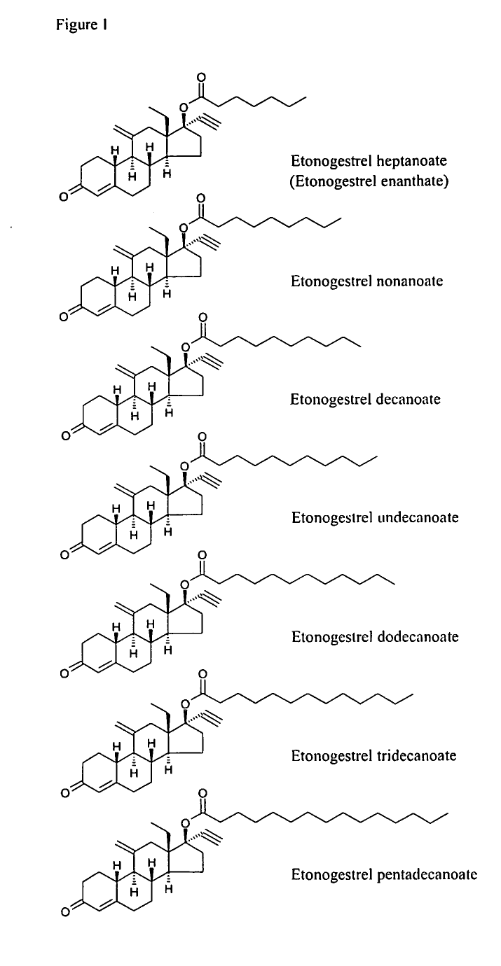

FIG. 1 shows the chemical structure of these compounds.

As a reference, etonogestrel was also included.

Preparation of Etonogestrel Esters

General methodology for the preparation of esters from alcohols can be found in e.g. Greene, T. W. et al, “Protective groups in organic synthesis”, John Wiley & Sons, NY, 1999 (third edition). Preparation of esters from tertiary alcohols (like etonogestrel) can be accomplished by several techniques, for instance:
1) tertiary alcohol, carboxylic acid, trifluoroacetic acid-anhydride, DE 1013284 (1956); 2) tertiary alcohol, acid chloride, pyridine, Watson, T. G. et al, Steroids 41, 255 (1983); 3) tertiary alcohol, acid chloride, TIOEt, Shafiee, A. et al, Steroids 41, 349 (1983), 4) tertiary alcohol, carboxylic acid-anhydride, TsOH, benzene, Johnson, A. L., Steroids, 20, 263 (1972); and 5) tertiary alcohol, carboxylic acid-anhydride, DMAP, CH₂Cl₂, Shafiee, A. et al, Steroids 41, 349 (1983).
   Preparation of (17α)-13-Ethyl-11-methylene-17-[[(1-oxononyl)oxy]-18,19-dinorpregn-4-en-20-yn-3-one (Etonogestrel Nonanoate)
a) A solution of nonanoic acid (1.95 g) in dry toluene (8 ml) was cooled to 0° C. and treated with trifluoroacetic acid anhydride (2.6 g). After 30 min. stirring, (17α)-13-ethyl-17-hydroxy-11-methylene-18,19-dinorpregn-4-en-20-yn-3-one (etonogestrel, 2.0 g) in dry toluene (15 ml) was added and the reaction mixture was stirred for 17 h at room temperature. The reaction mixture was washed with water, a saturated aqueous solution of sodium hydrogen carbonate, water, and brine. The organic phase was dried over sodium sulfate and concentrated under reduced pressure. The residue was purified by column chromatography (toluene/ethyl acetate 95:5). The product (2.08 g) was dissolved in ethyl acetate (40 ml), cooled to 0° C., and stirred with aqueous sodium hydroxide (1 M, 13 ml) for 2 h. The mixture was extracted with ethyl acetate; the combined organic phases were washed with ice-cold aqueous sodium hydroxide (1 M), water and brine, dried and concentrated under reduce pressure. Column chromatography afforded (17α)-13-ethyl-11-methylene-17-[[(1-oxononyl)oxy]-18,19-dinorpregn-4-en-20-yn-3-one (1.25 g). ¹H-NMR (CDCl₃): δ 5.89 (m, 1H), 5.08 (bs, 1H), 4.85 (bs, 1H), 2.82 (ddd, 1H, J=14.8, 9.5 and 6.3 Hz), 2.73 (d, 1H, J=12.8 Hz), 2.69-2.19 (m), 2.63 (s, 1H), 2.11 (m, 1H), 1.90-1.21 (m), 1.15 (m, 1H), 1.05 (t, 3H, J=7.5 Hz), 0.88 (t, 3H, J=7.1 Hz). Measured mass [M+H]⁺ 465.3358. Calculated mass [M+H]⁺ 465.3363.

In a manner analogous to the procedure described above, etonogestrel heptanoate, etonogestrel decanoate, etonogestrel undecanoate, etonogestrel dodecanoate, etonogestrel tridecanoate, and etonogestrel pentadecanoate were prepared:
b) (17α)-13-Ethyl-11-methylene-17-[[(1-oxoheptyl)oxy]-18,19-dinorpregn-4-en-20-yn-3-one (etonogestrel heptanoate). ¹H-NMR (CDCl₃): δ 5.89 (m, 1H), 5.08 (bs, 1H), 4.85 (bs, 1H), 2.82 (ddd, 1H, J=14.8, 9.5 and 6.3 Hz), 2.73 (d, 1H, J=12.6 Hz), 2.68-2.19(m), 2.63 (s, 1H), 2.11 (m, 1H), 1.90-1.24(m), 1.15 (m, 1H), 1.05 (t, 3H, J=7.5 Hz), 0.89 (t, 3H, J=7.1 Hz). Measured mass [M+H]⁺ 437.3027. Calculated mass [M+H]⁺ 437.3050.
c) (17α)-13-Ethyl-11-methylene-17-[[(1-oxodecyl)oxy]-18,19-dinorpregn-4-en-20-yn-3-one (etonogestrel decanoate). ¹H-NMR (CDCl₃): δ 5.89 (bs, 1H), 5.08 (bs, 1H), 4.84 (bs, 1H), 2.82 (m, 1H), 2.73 (d, 1H, J=12.6 Hz), 2.67-2.18 (m), 2.63 (s, 1H), 2.11 (m, 1H), 1.90-1.21 (m), 1.15 (m, 1H), 1.06 (t, 3H, J=7.5 Hz), 0.88 (t, 3H, J=7.1 Hz). Measured mass [M+H]+479.3508. Calculated mass [M+H]⁺ 479.3519.
d) (17α)-13-Ethyl-11-methylene-17-[[(1-oxoundecyl)oxy]-18,19-dinorpregn-4-en-20-yn-3-one (etonogestrel undecanoate). ¹H-NMR (CDCl₃): δ 5.89 (m, 1H), 5.08 (bs, 1H), 4.85 (bs, 1H), 2.82 (ddd, 1H, J=14.8, 9.5 and 6.3 Hz), 2.73 (d, 1H, J=12.6 Hz), 2.68-2.18 (m), 2.63 (s, 1H), 2.11 (m, 1H), 1.90-1.21 (m), 1.06 (t, 3H, J=7.5 Hz), 0.88 (t, 3H, J=7.1 Hz). Measured mass [M+H]⁺ 493.3664. Calculated mass [M+H]⁺ 493.3676.
e) (17α)-13-Ethyl-11-methylene-17-[[(1-oxododecyl)oxy]-18,19-dinorpregn-4-en-20-yn-3-one (etonogestrel dodecanoate). ¹H-NMR (CDCl₃): δ 5.89 (bs, 1H), 5.08 (bs, 1H), 4.85 (bs, 1H), 2.82 (m, 1H), 2.73 (d, 1H, J=12.6 Hz), 2.65-2.18 (m), 2.64 (s, 1H), 2.11 (m, 1H), 1.90-1.20 (m), 1.15 (m, 1H), 1.06 (t, 3H, J=7.5 Hz), 0.88 (t, 3H, J=7.1 Hz). Measured mass [M+H]⁺ 507.3829. Calculated mass [M+H]⁺ 507.3832.
f) (17α)-13-Ethyl-11-methylene-17-[[(1-oxotridecyl)oxy]-18,19-dinorpregn-4-en-20-yn-3-one (etonogestrel tridecanoate). ¹H-NMR (CDCl₃): δ 5.89 (bs, 1H), 5.08 (bs, 1H), 4.85 (bs, 1H), 2.82 (m, 1H), 2.73 (d, 1H, J=12.6 Hz), 2.65-2.18 (m), 2.64 (s, 1H), 2.11 (m, 1H), 1.90-1.20 (m), 1.15 (m, 1H), 1.06 (t, 3H, J=7.5 Hz), 0.89 (t, 3H, J=7.1 Hz). Measured mass [M+H]⁺ 521.4007. Calculated mass [M+H]⁺ 521.3989.
g) (17α)-13-Ethyl-11-methylene-17-[[(1-oxopentadecyl)oxy]-18,19-dinorpregn-4-en-20-yn-3-one (etonogestrel pentadecanoate). ¹H-NMR (CDCl₃): δ 5.89 (bs, 1H), 5.08 (bs, 1H), 4.85 (bs, 1H), 2.82 (m, 1H), 2.73 (d, 1H, J=12.6 Hz), 2.65-2.19 (m), 2.63 (s, 1H), 2.11 (m, 1H), 1.90-1.20 (m), 1.15 (m, 1H), 1.06 (t, 3H, J=7.5 Hz), 0.89 (t, 3H, J=7.1 Hz). Measured mass [M+H]⁺ 549.4278. Calculated mass [M+H]⁺ 549.4302.
   Pharmacokinetics Studies in the Rabbit

For the determination of the pharmacokinetic profile of the different etonogestrel-esters after parenteral application, i.m. application in the castrated rabbit model was chosen instead of s.c. Briefly, rabbits were injected once (day 1) with indicated etonogestrel-esters at 20 mg/kg in arachis oil (with a concentration of 40 mg/ml). At day 1, 2, 3, 4, 5, 6, 7, 8, 10, 12, 14, 21, 28, 35, 49, 63, 77, 92, 106, 120 and 133 blood was collected from the ear arteria, in EDTA-containing tubes. EDTA plasma was prepared (1500 g, 15 min) and stored at −20° C. With LC-MSMS the amount of parent compound (etonogestrel) was determined in these samples. The lower limit of this new assay is 0.5 nmol/l, from 0-250 nmol/l a linear curve was obtained with a correlation coefficient of 0,9998.

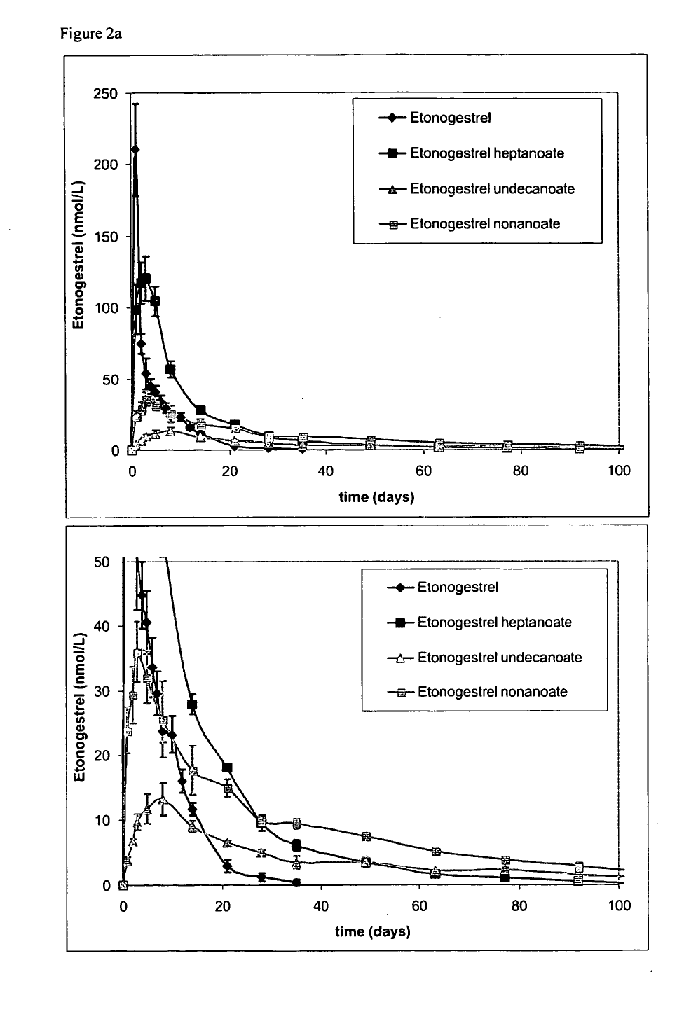

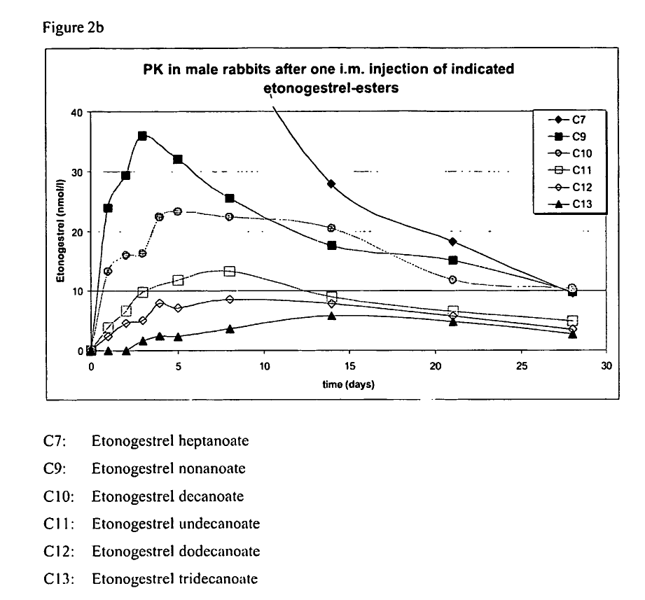

As shown in FIG. 2a, etonogestrel itself resulted in very high peak levels (200 nmol/l), which declined in 28 days to levels of etonogestrel below 1 nmol/l.

Etonogestrel-heptanoate also gave rise to high initial peak levels of etonogestrel (120 nmol/l). Etonogestrel nonanoate gave lower peak levels and extended duration with serum levels of etonogestrel above 1 nmol/l. As compared to the other two esters in FIG. 2a, etonogestrel undecanoate gave the most optimal balance between initial peak levels (maximum of 13 nmol/l after eight days) and duration of action (more than 92 days above 1 nmol/l).

As shown in FIG. 2b, etonogestrel decanoate gave an initial peak level of 24 nmol/l after 5 days whereas etonogestrel dodecanoate gave an initial peak level of 9 nmol/l after 8 days. With etonogestrel tridecanoate, no initial levels of etonogestrel were observed.

From FIGS. 2a and 2b, it can be seen that preferred etonogestrel esters are etonogestrel decanoate, etonogestrel undecanoate, and etonogestrel dodecanoate.

### Example 2

### Kinetics of Two MENT Esters in Rabbits

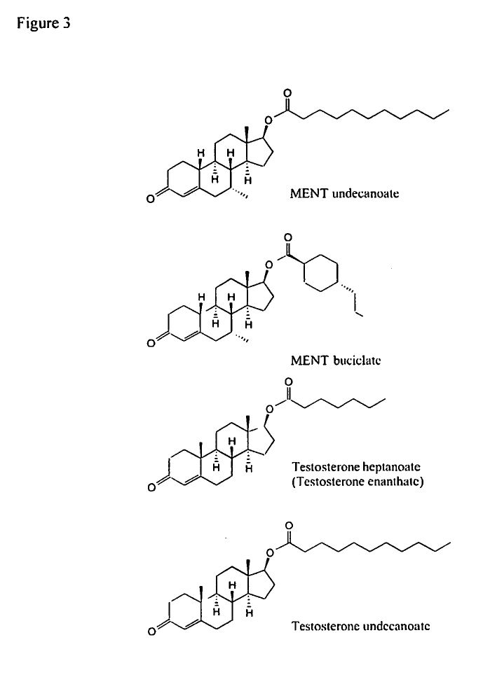

The pharmacokinetic profile of MENT-undecanoate and MENT-buciclate was compared to testosterone-enanthate and testosterone-undecanoate. FIG. 3 shows the chemical structures of these androgen esters.

Ment-undecanoate was prepared essentially as described in WO 99/67271. MENT-buciclate was prepared as described in WO 99/67270. Testosterone enanthate and undecanoate were commercially obtained from Diosynth, Oss, the Netherlands.

Pharmacokinetic Studies in the Rabbit

For the determination of the pharmacokinetic profile of the different androgen-esters after s.c. application, the castrated rabbit model was selected as the model which is most similar to humans. Briefly, rabbits were injected once (day 1) with indicated androgen-esters at 20 mg/kg in arachis oil (with a concentration of 100 mg/ml). At day 2, 3, 4, 5, 8, 15, 22, 36, 44 and 58 blood was collected from the ear arteria, in EDTA-containing tubes. EDTA plasma was prepared (1500 g, 15 min) and stored at −20° C. With LC-MSMS, the amount of parent compound (testosterone or MENT) was determined in these samples. The lower limit of this new assay is 2 nmol/l, from 0-500 nmol/l a linear curve was obtained with a correlation coefficient of 0,9998.

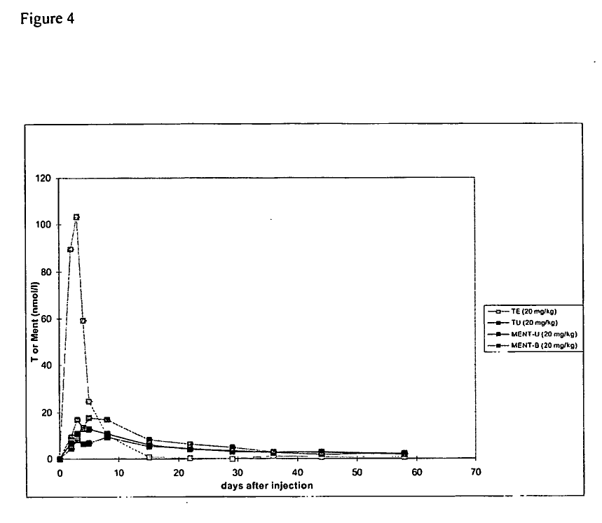

As shown in FIG. 4, both with MENT-undecanoate and MENT-buciclate a pharmacokinetic profile of released MENT was found which is similar to that of the reference compound testosterone-undecanoate with respect to released testosterone. Testosterone-enanthate resulted in a high peak of testosterone 2 days after injection.

Thus, in the rabbit, with both MENT-esters no initial rise of MENT was observed on one hand and a prolonged release of MENT was observed on the other hand, suggestive for more optimal pharmacokinetic behaviour than the current standard testosterone-enanthate.

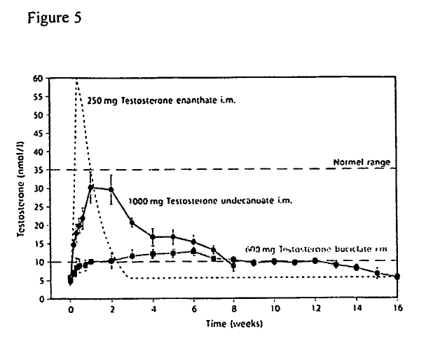

In humans, optimal pharmacokinetics were obtained with testosterone undecanoate: low initial release and steady-state levels of long duration (FIG. 5). Since in rabbits the pharmacokinetic profile of the two MENT-esters was very similar to that of testosterone-undecanoate (FIG. 4), optimal pharmacokinetics with both MENT esters in humans is expected.

### Example 3

### Solubility and Viscosity of MENT-Undecanoate and Etonogestrel Undecanoate in Various Solvents

To determine the solubility and viscosity of MENT undecanoate and etonogestrel undecanoate, four different solvents were used:
ethyl undecanoate
ethyl undecanoate+50% benzyl benzoate
arachis oil
arachis oil+50% benzyl benzoate

Using these solvents, the following solutions were prepared:
100 mg/ml etonogestrel undecanoate in the different solvents
50 mg/ml etonogestrel undecanoate in the different solvents
200 mg/ml MENT undecanoate in the different solvents
100 mg/ml MENT undecanoate in the different solvents
50 mg/ml etonogestrel undecanoate+100 mg/ml MENT undecanoate in the different solvents

The two combined solvents were prepared by addition of 50 gram of ethyl undecanoate or arachis oil to 50 gram of benzyl benzoate. The ethyl undecanoate+50% benzyl benzoate solution was filtered over a 0.22 μm Durapore filter to obtain a clear colourless solution. The arachis oil+50% benzyl benzoate solution was not filtered.

The solubility of the compounds in the solvents was determined visually. The viscosity was determined using a Brookfield model DV-III. The density of the solutions was determined using a Mettler Toledo DA-100M density meter.

TABLE 1
Appearance, viscosity and density of the solvents

| Solvent | Appearance | Viscosity | Density |
|---|---|---|---|
| Ethyl undecanoate | Clear colourless | 2.6 | 0.861 |
| | solution | | |
| Ethyl undecanoate + | Clear colourless | 3.9 | 0.975 |
| 50% benzyl benzoate | solution | | |
| Arachis oil | Clear yellowish | 64.1 | 0.913 |
| | solution | | |
| Arachis oil + 50% | Clear yellowish | 22.9 | 1.007 |
| benzyl benzoate | solution | | |
| Benzyl benzoate | Clear yellowish | 8.5 | 1.117 |
| | solution | | |

Ethyl undecanoate, ethyl undecanoate+50% benzyl benzoate and arachis oil+50% benzyl benzoate solutions did not need to be heated. To dissolve 200 mg/ml MENT undecanoate in arachis oil, heating to approximately 50° C. was necessary.

The concentrations tested were 100 mg/ml etonogestrel undecanoate, 200 mg/ml MENT undecanoate and 50 mg/ml etonogestrel undecanoate+100 mg/ml MENT undecanoate in the different solvents. The results are summarized in table 2.

TABLE 2
Appearance, viscosity and density of the final solutions

| | Etonogestrel | MENT | | | |
|---|---|---|---|---|---|
| | undecanoate | undecanoate | | Viscosity | Density |
| Solvent | (mg/ml) | (mg/ml) | Appearance | (cps) | (g/ml) |
| Ethyl undecanoate | 50 | — | Clear colourless solution | 3.2 | 0.870 |
| | — | 100 | Clear colourless solution | 4.0 | 0.879 |
| | 50 | 100 | Clear colourless solution | 4.4 | 0.886 |
| Ethyl undecanoate + | 50 | — | Clear colourless solution | 4.7 | 0.978 |
| 50% benzyl | — | 100 | Clear colourless solution | 6.1 | 0.979 |
| benzoate | 50 | 100 | Clear colourless solution | 7.0 | 0.979 |
| Arachis oil | 50 | — | Clear yellowish solution | 76.6 | 0.919 |
| | — | 100 | Clear yellowish solution | 97.2 | 0.924 |
| | 50 | 100 | Clear yellowish solution | 99.7 | 0.935 |
| Arachis oil + 50% | 50 | — | Clear yellowish solution | 28.1 | 1.006 |
| benzyl benzoate | — | 100 | Clear yellowish solution | 35.0 | 1.009 |
| | 50 | 100 | Clear yellowish solution | 39.1 | 1.008 |

The combination of etonogestrel-undecanoate and MENT-undecanoate was visually dissolved at a desired concentration of 50 mg/ml etonogestrel-undecanoate and 100 mg/ml MENT-undecanoate in all four tested solvents. Both etonogestrel-undecanoate and MENT-undecanoate could be dissolved at two times the desired concentration in all four solvents tested. No precipitation occurred at room temperature when 50 mg/ml etonogestrel-undecanoate and 100 mg/ml MENT-undecanoate were dissolved in all four solvents.

The viscosity of ethyl undecanoate and ethyl undecanoate+50% benzyl benzoate was significantly lower than the viscosity of arachis oil and arachis oil+50% benzyl benzoate. The viscosity of the desired formulation 50 mg/ml etonogestrel undecanoate+100 mg/ml MENT undecanoate in the four different solvents was the lowest (4 cps) for the ethyl undecanoate solution, followed by the ethyl undecanoate+50% benzyl benzoate (7 cps) and the arachis oil+50% benzyl benzoate solution (39 cps). The viscosity of the arachis oil solution was significantly higher that the viscosity of the other solutions (100 cps).

### Example 4

### Pharmacological Action of Etonogestrel Esters in the Male

The pharmacological action of etonogestrel esters in the male are evaluated for the suppressing activity of endogenous testosterone in the rabbit as described in Wu, F. C., Balasubramanian, R., Mulders, T. M. and Coelingh-Bennink H. J., Oral progestogen combined with testosterone as a potential male contraceptive: additive effects between desogestrel and testosterone enanthate in suppression of spermatogenesis, pituitary-testicular axis, and lipid metabolism, J. Clin. Endocrinol. Metab 84 (1):112-122, 1999. Briefly, the effect of one sc/im injection of the different etonogestrel esters on serum testosterone at day 7 of mature male rabbits will be monitored.

### Example 5

### The Pharmacological Action of Etonogestrel Esters in the Female

The pharmacological action of etonogestrel esters in the female are tested in the classical Clauberg test. Briefly, immature female rabbits, primed with oestradiol for 8 days, are treated once sc/im with the different etonogestrel esters (day 8 afternoon). Autopsy is performed in the afternoon of day 13 and the progestagenic activity is evaluated on sections of the uterine according to McPhail et al., The assay of progestin. J. of Physiology, 1934, 83:145-156.

1. A contraceptive and/or HRT kit comprising a contraceptively and/or therapeutically effective amount of a long-acting etonogestrel ester with a fatty-chain length of C7-C15.

2. The kit according to claim 1, wherein the etonogestrel ester is etonogestrel undecancate and/or etonogestrel decanoate and/or etonogestrel dodecanoate.

3. The kit according to claim 1, further comprising a contraceptively and/or therapeutically effective amount of an androgen ester.

4. The kit according to claim 3, wherein the androgen ester is MENT undecanoate.

5. The kit accordhig to claim 4, wherein the contraceptively and/or therapeutically effective amount of MENT undecanoate is 50-400 mg and the contraceptively and/or therapeutically effective amount of etonogestrel ester is 25-200 mg.

6. The kit according to claim 5, wherein the contraceptively and/or therapeutically effective amount of MENT undecanoate is 50-200 mg and the contraceptively and/or therapeutically effective amount of etonogestrel ester is 50-100 mg.

7. The kit according to claim 6, wherein the contraceptively and/or therapeutically effective amount of MENT undecanoate is 100 mg and the contraceptively and/or therapeutically effective amount of etonogestrel ester is 50 mg.

8. The kit according to claim 1, further comprising a contraceptively and/or therapeutically effective amount of an estrogen.

9. (canceled)

10. (canceled)

11. (canceled)

12. (canceled)

13. (canceled)

14. (canceled)

15. (canceled)

16. (canceled)

17. (canceled)

18. A method of contraception and/or HRT, comprising:
administering to a subject a contraceptively and/or therapeutically effective amount of a long acting etonogestrel ester with a fatty-chain length of C7-C15.

19. The method according to claim 18, wherein the ester is etonogestrel undecanoate and/or etonogestrel decanoate and/or etonogestrel dodecanoate.

20. The method according to claim 18, wherein a contraceptively and/or therapeutically effective amount of a long-acting androgen ester is administered in conjunction with the etonogestrel ester.

21. The method according to claim 20, wherein the etonogestrel ester is etonogestrel undecanoate and/or etonogestrel decanoate and/or etonogestrel dodecanoate and the androgen ester is MENT undecanoate.

22. The method according to claim 21, wherein the contraceptively and/or therapeutically effective amount of MENT undecanoate is 50-400 mg and the contraceptively and/or therapeutically effective amount of etonogestrel ester is 25-200 mg.

23. The method according to claim 22, wherein the contraceptively and/or therapeutically effective amount of MENT undecanoate is 50-200 mg and the contraceptively and/or therapeutically effective amount of etonogestrel ester is 50-100 mg.

24. The method according to claim 23, wherein the contraceptively and/or therapeutically effective amount of MENT undecanoate is 100 mg and the contraceptively and/or therapeutically effective amount of etonogestrel ester is 50 mg.

25. The method according to claim 18, wherein a contraceptively and/or therapeutically effective amount of an estrogen is administered in conjunction with the etonogestrel ester.

26. (canceled)

27. (canceled)

28. (canceled)

29. A method of treating and/or preventing a female gynecological disorder, comprising:
administering to a female subject a therapeutically effective amount of a long-acting etonogestrel ester with a fatty-chain length of C7-C15 effective to treat and/or prevent the disorder.

30. The method according to claim 29, wherein the etonogestrel ester is etonogestrel undecanoate and/or etonogestrel decanoate and/or etonogestrel dodecaneate.

31. The method according to claim 29, wherein the female gynecological disorder is selected from the group consisting of endometriosis, menorrhagia, meno-metrorrhagia, pre-menstrual syndrome and dysmenorrhoea.
